(19) United States
(12) Plant Patent Application Publication
(10) Pub. No.: US 2018/0324993 P1
(43) Pub. Date: Nov. 8, 2018

(54) Strawberry plant named 'Octavia'

(50) ;

(71) Applicant: SWEET DARLING SALES, INC., Aptos, CA (US)

(72) Inventor: John Larse, Watsonville, CA (US)

(21) Appl. No.: 15/932,839

(22) Filed: May 4, 2018

Related U.S. Application Data
(60) Provisional application No. 62/501,253, filed on May 4, 2017.

(51) Int. Cl. A01H 6/74

(52) U.S. Cl. CPC A01H 6/7409
USPC PLT/208

(57) ABSTRACT

The present invention provides new and distinct strawberry plant designated as ‘Octavia’ (a.k.a. ‘108991’).

### CROSS-REFERENCE TO RELATED APPLICATION

The present application claims priority to, and the benefit of U.S. Provisional Patent Application No. 62/501,253, filed on May 4, 2017, which is herein incorporated by reference in its entirety.

Latin name of the genus and species: Fragaria x ananassa.

Varietal denomination: ‘Octavia’ (a.k.a. ‘108991’).

### BACKGROUND OF THE INVENTION

The present invention relates to new and distinct strawberry plant designated as ‘Octavia’ (a.k.a. ‘108991’).

‘Octavia’ (a.k.a. ‘108991’) is the result of a controlled-cross between a female parent cultivar designated 107967 and a male parent cultivar designated 3236 made by the Inventor and was first fruited in Watsonville, Calif. growing fields. Following selection and during testing, the plant was originally designated ‘108991’ and subsequently named ‘Octavia’. ‘Octavia’ is a day-neutral plant.

This new strawberry plants was asexually reproduced via runners (stolons) by the inventor at Watsonville, Calif. Asexual propagules from the original source have been tested in Watsonville growing fields and to a limited extent, grower fields in high elevation. The properties of this plant were found to be transmissible by such asexual reproduction. The plant is stable and reproduces true to type in successive generations of asexual reproduction.

### BRIEF SUMMARY OF THE INVENTION

This invention relates to new and distinctive strawberry plant designated as ‘Octavia’ (a.k.a. ‘108991’). The plant is primarily adapted to the climate and growing conditions of the central coast of California. This region provides the necessary temperatures required for it to produce a strong vigorous plant and to remain in fruit production from March through October. The nearby Pacific Ocean provides the needed humidity and moderate day temperatures and evening chilling to maintain fruit quality for the production months.

The following traits and photographs in combination distinguish strawberry plant ‘Octavia’ from known strawberry plants. Plants for the botanical measurements in the present application were grown as annuals. Any color references are made to The Royal Horticultural Society Colour Chart, 1995 Edition, except where general terms of ordinary dictionary significance are used.

TABLE 1
‘Octavio’

| | Characteristic | Description |
|---|---|---|
| General | Plant Habit | perennial |
| | Plant Growth Habit | semi-upright |
| | Height (cm) | 39.5 cm |
| | Width (cm) | 35 cm |
| | Density of foliage, vigor | light |
| | Plant vigor | moderate to high |
| Leaf | Terminal leaflet width (mm) | 85.88 mm |
| | Terminal leaflet length (mm) | 78.6 mm |
| | No. teeth/terminal leaflet: | 21 |
| | shape of the terminal leaflet base | acute |
| | shape of terminal leaflet in cross-section | concave |
| | margin description of the terminal | crenate |
| | Color of upper side of leaves | 137A |
| | Color of lower side of leaves | 138D |
| | Leaf blistering | weak |
| | Leaf glossiness | medium |
| Limbs | Petiole length (cm) | 24.5 mm |
| | Petiole diameter (cm) | 4.57 mm |
| | Petiole color | 145A |
| | Petiolule length (cm) | 9.84 mm |
| | Petiolulc diamctcr (cm) | 2.18 mm |
| | Stipule length (cm) | 3.5 cm |
| | Stipule width (cm) | 11 mm |
| | Stipule anthocyanin | present |
| | Stipule color (color code) | 145A |
| | Pedicel color (color code) | 145A |
| Limbs | attitude of hairs on | upwards |
| | petiole and pedicel | |
| Inflorescence | Inflorescence position relative to foliage | above |
| | flower arrangement of petals | free to touching |
| | Flower diameter (cm) | 14.05 mm |
| | Petal length (cm) | 15.23 mm |
| | Petal width (cm) | 5 to 6 |
| | Petal number per flower | 155C |
| | Upper and lower Petal color | 155C |
| | peduncle size | medium |
| | Calyx diameter (cm) | 28.28 mm |
| | Corolla diameter (cm) | 32.60 mm |
| | Sepal length (cm) | 8.95 mm |
| | Sepal width (cm) | 6.11 mm |
| | Number of stamen | 27 |
| | Anther color | 12A |
| | Time of flowering | April |
| | (50% of plants in bloom) | |
| | Shape of stigma | capitate |
| | Color of stigma | 14A |
| | Length of style | 2.5 mm |
| | Color of style | 12A |
| | Color of the ovary | 145C |
| | Length of the stamens | 3.13 mm |
| | Shape of anther | dorsifixed |
| | Size of anther | 1.74 mm |
| | Color of anther | 12A |
| | Amount of pollen | scarce to moderate |
| | Color of pollen | 163A |
| | Color of filament | 145C |
| | Length of filament | 3.92 mm |
| Stolon | Stolon number | 2 to 5 |
| | Stolon anthocyanin | 181A |
| | Widest diameter of stolon | 6.17 mm |
| | At leaf attachment | |
| | Stolon color | 145B |
| Fruit | Number of fruit per truss | 2 to 5 |
| | Fruit length (cm) | 4.8 cm |
| | Fruit width (cm) | 4.1 cm |
| | Fruit skin color | 45A |
| | Fruit flesh color excluding core | 41B |
| | Fruit core color | 39B |
| | Fruit weight (g) | 29 g |
| | Predominant fruit shape | long conic |
| | Shape difference between primary | No shape |
| | & secondary fruits | difference |
| | Width of band without of achenes | medium |
| | Fruit glossiness | strong |
| | Position of achenes | below |
| | Achene color | 2C |
| | Achenes per fruit | 475 |
| | Achene weight (g) | 0.28 g |
| | Position of calyx | above |
| | Diameter of calyx | 3.3 cm |
| | level of adherence of calyx | medium |
| | Color of calyx | 145B |
| | Firmness of flesh | firm |
| | Evenness of flesh color | nearly even |
| | Sweetness (Brix) | 7 |
| | pH | 3.44 |

‘Octavia’ is similar to the UC Davis variety ‘Monterey’ (U.S. Plant Pat. No. 19,767), but possesses several distinguishing traits from ‘Monterey’. ‘Octavia’ presents shorter petiole lengths and flower stem lengths than ‘Monterey’. ‘Monterey’ produces mostly conic and long conic fruit while ‘Octavia’ produces conic, long conic, short wedge and long wedge fruit. The male parent of ‘Octavia’ differs from ‘Octavia’ by producing globose fruit. Both the female parent of ‘Octavia’ and ‘Monterey’ produce firmer fruit than ‘Octavia’. ‘Octavia’ produces larger strawberries than both of its parents do.

### DESCRIPTION OF THE DRAWINGS

The accompanying color photographs depict various characteristics of the cultivars as nearly true as possible to make color reproductions.

FIG. 1 shows ripe and near-ripe fruits of ‘Octavia’ about 6-month old.

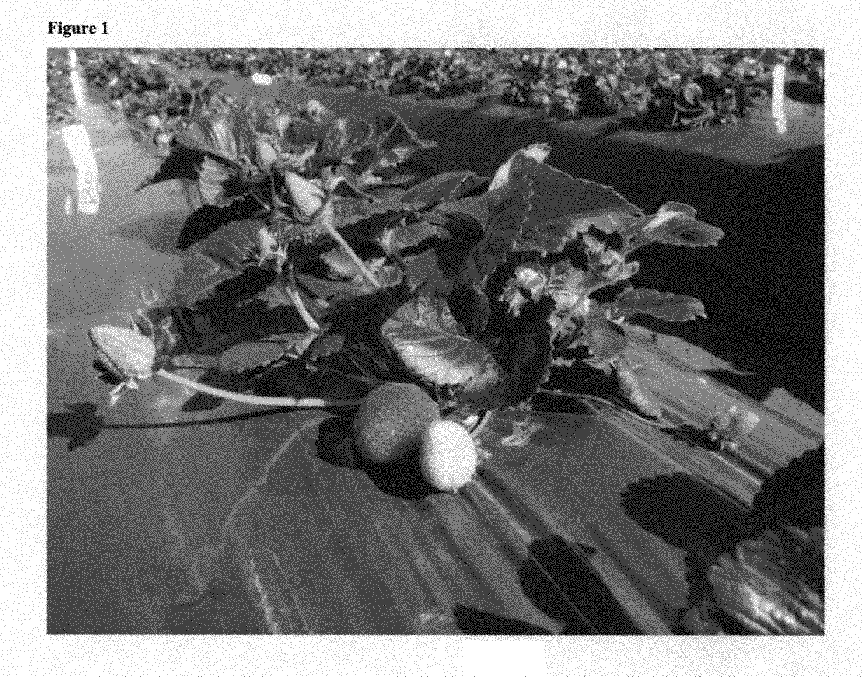

FIG. 2 shows a leaf of ‘Octavia’ plants.

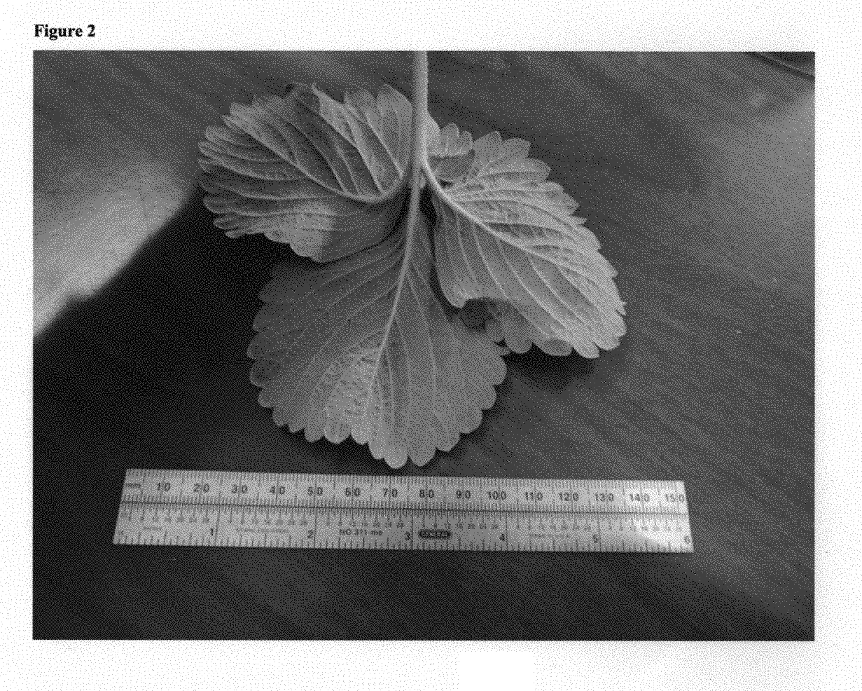

FIG. 3 shows petiole of ‘Octavia’ plants.

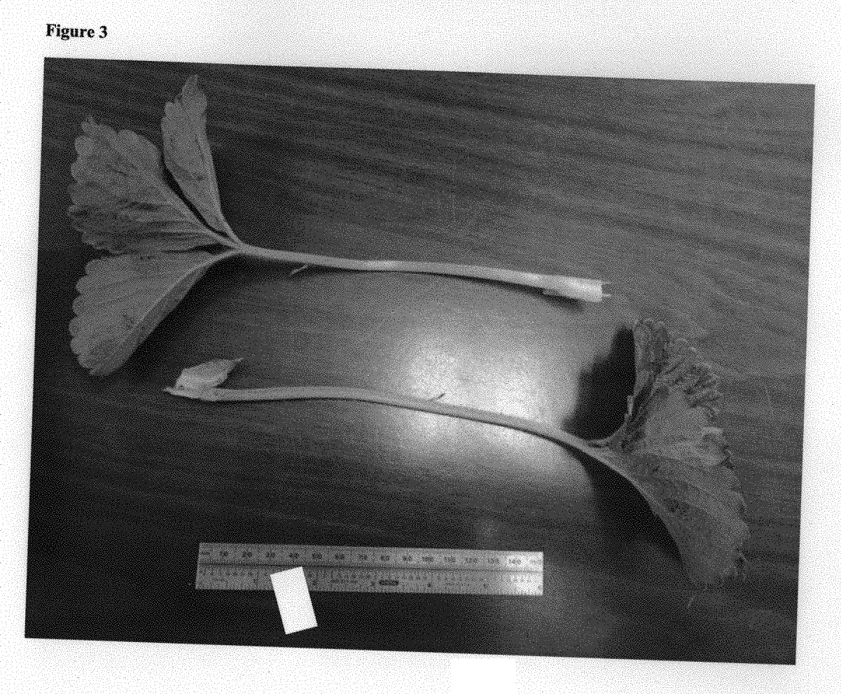

FIG. 4 shows inflorescences of ‘Octavia’ plants.

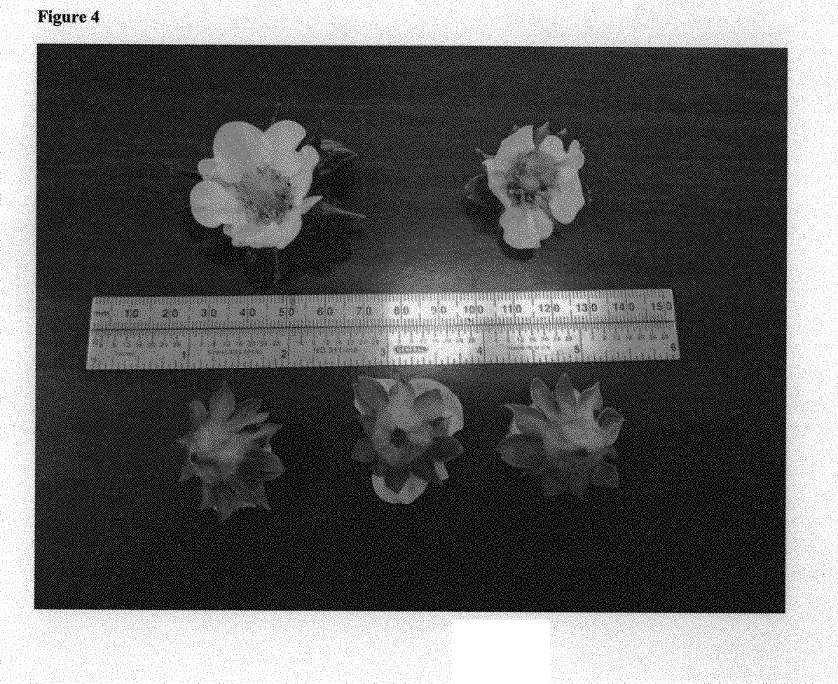

FIG. 5 shows fruits of ‘Octavia’ plants.

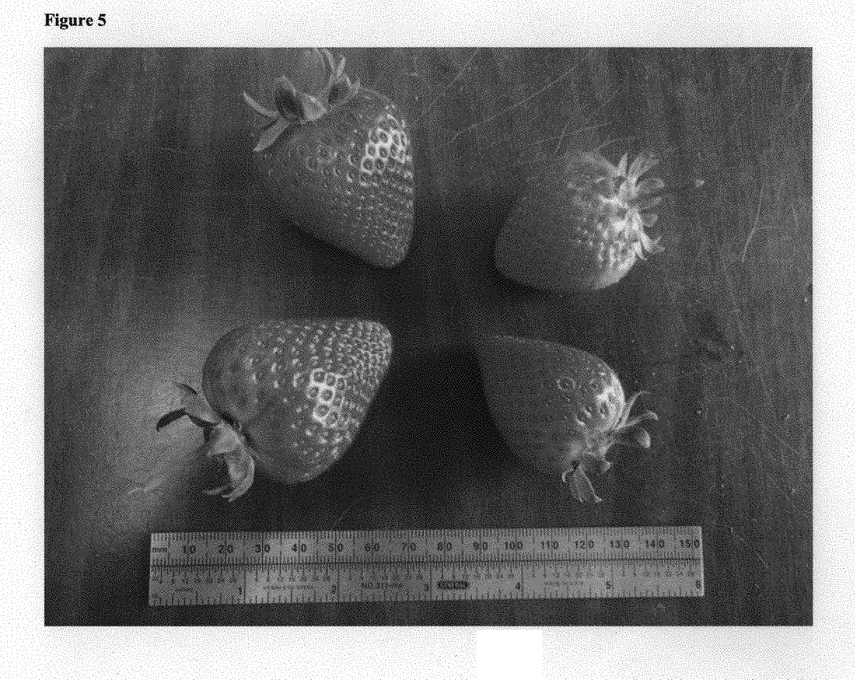

FIG. 6 shows fruits of ‘Octavia’ plants cut in half.

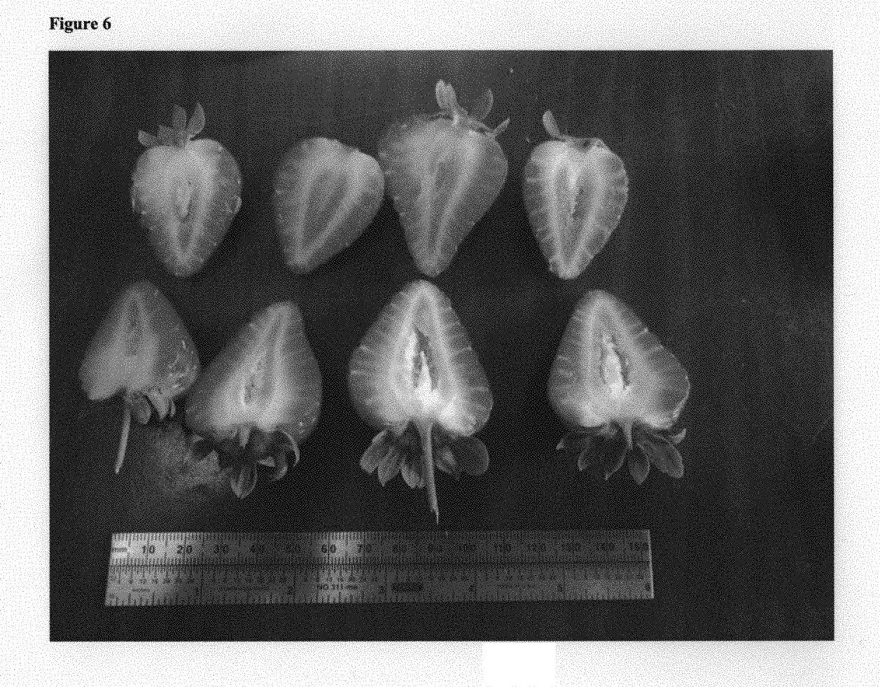

1. A new and distinct cultivar of strawberry plant named ‘Octavia’, substantially as shown and described herein.
